(19) United States
(12) Patent Application Publication
(10) Pub. No.: US 2005/0004455 A1
(43) Pub. Date: Jan. 6, 2005

(54) Method and apparatus for controlling the radiation dose in the generation of x-ray images for lithotripsy

(75) Inventor: Rainer Kaltschmidt, Eckental/Brand (DE)

Correspondence Address: SCHIFF HARDIN LLP;Patent Department, 6600 Sears Tower, 233 South Wacker Drive, Chicago, IL, 60606, US

(73) Assignee: Siemens Aktiengesellschaft

(21) Appl. No.: 10/882,957

(22) Filed: Jul. 1, 2004

(30) Foreign Application Priority Data
Jul. 1, 2003 (DE) 103 29 299.3

(51) Int. Cl. A61B 5/05; A61B 17/22

(52) U.S. Cl. 600/427; 601/4

(57) ABSTRACT

In a method and an apparatus to generate an x-ray image from the focus region of a lithotripter using x-rays that pass through the focus region and device parts of the lithotripter, to control the dose of the x-rays, only an image region of the image field exposed by the x-rays, that lies outside of an area shadowed by these device parts, is evaluated.

### BACKGROUND OF THE INVENTION

1. Field of the Invention

The present invention concerns a method for controlling the radiation dose in the generation of an x-ray image from the focus region of a lithotripter, and an apparatus to implement the method.

2. Description of the Prior Art

Lithotripsy is a therapeutic method to destroy (without a surgical procedure) a calculus located in the body of an organism using a focused ultrasound shockwave that is generated by a shockwave generator. In order to largely prevent damages in the tissue surrounding the calculus, it is necessary on the one hand to position the focus of the lithotripter exactly in the calculus. It is also necessary to visualize the progress in the destruction of the calculus in order to limit the number of shockwave pulses to the therapeutically necessary minimum.

To generate an x-ray image of the environment of the focus region of a lithotripter, it is known, for example, from German OS 197 46 956 to couple an x-ray apparatus with a lithotripter such that the x-rays generated by the x-ray apparatus pass through the focus region of the lithotripter. This apparatus is known as an “off-line” or “out-line” arrangement in which the center axis of the x-ray radiation is disposed inclined relative to the center axis of the focused shockwave generated by the shockwave generator, and the focus region is located approximately in the intersection point of these center axes.

As an alternative or supplement to this, it is also known to generate an x-ray image from the focus region using a so-called “in-line” arrangement, in which the center axis of the x-rays generated for image reproduction coincides with the center axis of the focused shockwave.

During such a lithotripsy treatment, it is necessary to acquire a number of x-ray images both for exact positioning of the focus of the shockwave and for visualization of the process of the disintegration of the calculus or calculi. In spite of the use of x-ray image intensifiers and the use that is thereby made possible of x-ray sources with a relatively low dose, as before it is desirable to further reduce the dose (exposure) of the patient.

### SUMMARY OF THE INVENTION

An object of the present invention to provide a method to generate an x-ray image from the focus region of a shockwave generator with which a reduction of the dose load to a patient is enabled. A further object of the invention is to provide an apparatus operating according to this method.

The above object is achieved in accordance with the present invention by a method for controlling the radiation dose in the generation of x-ray images for lithotripsy, wherein an x-ray image is generated by x-rays passing through the focus region of a lithotripter having device components, wherein at least one of the device components produces a region in the total x-ray exposure that is shadowed by that component, and wherein only an image region in the exposure that lies outside of this shadowed region is evaluated for controlling the radiation dose.

Since, to control the dose of the x-ray radiation, only one image region of the image field exposed by the x-ray radiation is evaluated, the image field being outside of an area shadowed by apparatus parts of the lithotripter, it is possible to optimize the image quality and therewith the radiation dose for this image region.

The invention is based on the realization that shadowed and diagnostically unusable image regions unavoidably exist in x-ray images from the focus region of a lithotripter. In an “out-line” arrangement this component is the partially imaged coupler bellows (cushion), (through which a portion of the x-ray radiation passes) of the shockwave generator that effects an attenuation of the x-ray radiation. In an “in-line” arrangement, this component is the surroundings of an aperture diaphragm used for the x-ray radiation in the coupler bellows, and the additional water flow line, which are traversed by the x-ray radiation. These items reduce the brightness of the x-ray image with respect to the total image field and cause the automatic dose control used in the prior art to both increase the dose rate and the operating voltage of the x-ray tube. In addition to an increase of the dose, due to the lesser primary absorption of higher-energy x-ray quanta, this additionally leads to a worsening of the image contrast and thus the recognizability of the calculus.

To control the x-ray system, the invention utilizes for exposure control only those parts of the total image field that are diagnostically usable and of paramount interest, and that preferably lie outside of a shadowed area and that includes the immediate surroundings of the focus of the lithotripter. As a result, a specific underexposure (measured on the average brightness of the total image field) occurs that is nevertheless sufficient to show the primary diagnostically interesting portion of the total image field in good image quality. As a result of this measure, the radiation exposure of the patient is decidedly reduced without leading to a worsening of the reproduction quality in these diagnostically interesting regions.

In an embodiment of the method, the shadowed area of the image field is lightened with a digital image processing method. In spite of the underexposure, it is possible to still diagnostically utilize this area for locating the stone.

The above object also is achieved in accordance with the present invention by an apparatus for implementing the method described above.

### DESCRIPTION OF THE DRAWINGS

FIG. 1 is a block diagram of an apparatus according to the invention.

FIGS. 2 and 3 respectively show an advantageous selection of the image region used for evaluation given an “in-line” arrangement (FIG. 2) and an “out-line” arrangement (FIG. 3).

### DESCRIPTION OF THE PREFERRED EMBODIMENTS

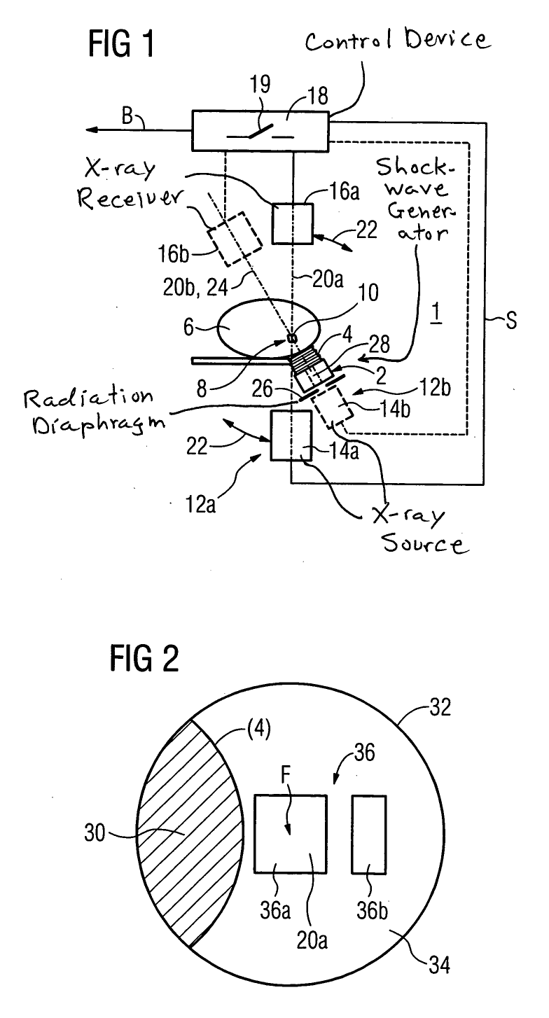

An apparatus according to the invention and shown schematically in FIG. 1 has a lithotripter 1 with a shockwave generator 2 placed over a coupler bellows 4 filled with water on the surface of the body 6 of a living subject. The shockwave generator 2 generates a shockwave that is focused in a focus region 8. Given correct positioning of the shockwave generator 2, a calculus 10 to be destroyed is located in this focus region 8.

An x-ray apparatus 12a that has an x-ray source 14a as well as an x-ray receiver 16a is associated with the lithotripter 1. A control signal S for inventive contrast-optimized and dose-optimized control of the x-ray source 14a is generated from the x-ray image acquired by the x-ray receiver 16a after digitization and evaluation. An optimized x-ray image B generated in this manner is output and, for example, reproduced on a monitor. With a selector switch 19 symbolically illustrated in FIG. 1, the user can decide whether a contrast-optimized and dose-optimized control of the x-ray source 14a should ensue according to the invention, or whether a conventional control is temporarily desired in order to also diagnostically use the shadowed regions.

The center axis 20a of the x-ray radiation emitted by the x-ray source 14a, meaning the connecting line between the x-ray source 14a and the x-ray receiver 16a, is positioned such that it intersects the focus region 8 of the lithotripter. In other words, the x-rays emitted by the x-ray source 14a pass through the focus region 8 of the lithotripter. These x-ray source 14a and the x-ray receiver 16a can be mounted on a C-arm (symbolically illustrated in FIG. 1 by double arrow 22) and are both pivoted around the intersection point between the center axis 20a of the x-ray radiation and the center axis 24 of the shockwave emitted by the shockwave generator 2.

The x-ray apparatus 12a shown in FIG. 1 is the type known as an “out-line” arrangement in which the center axis 20a of the x-ray radiation and the center axis 24 of the shockwave are inclined toward one another, meaning they do not coincide.

As an alternative or supplement to the x-ray apparatus 12a, an x-ray apparatus 12b is indicated dashed in FIG. 1 of the type known as an “in-line” arrangement in which center axis 20b of the x-ray radiation and center axis 24 of the ultrasound shockwave coincide. A diaphragm 26 (shown schematically in FIG. 1) in front of the x-ray source 16b limits the transmitted x-ray radiation to a central region around the center axis 20b or 24. As shown in FIG. 1, in an “in-line” arrangement it is typical to dispose the x-ray source 14b behind the shockwave generator 2 provided with a penetration opening. This has the advantage that the unavoidable additional absorption of the x-ray radiation (due to the water flow line) in the region of the center axis 24, and the higher dose that is therefore necessary, does not lead to an increased radiation exposure of the patient, since the absorption occurs before the x-rays enter into the body 6. Nevertheless, in this case a modulation of the x-ray system according to the prior art would lead to an overexposure of the central region and an unwanted contrast reduction, due to the higher-energy x-ray quanta. However, in principle the positions of the x-ray source 14b and the x-ray receiver 16b can be exchanged, such that the x-ray receiver 16b is behind the shockwave generator 2. In this case, the diaphragm 26 is also arranged directly in front of the x-ray source 14b.

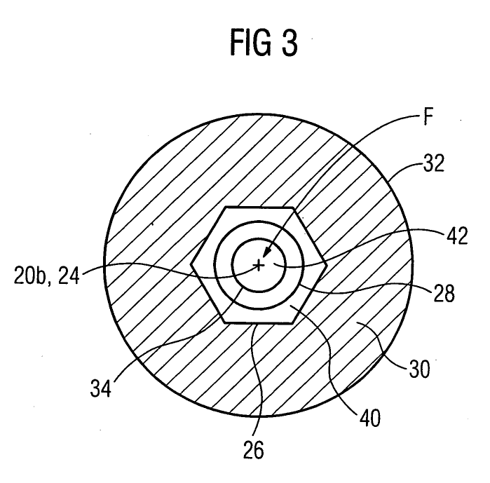

According to FIG. 2, the x-ray image acquired by the x-ray receiver 16a now has a structure that appears similar to a “three-quarter moon”. In FIG. 2, it is indicated by hatching that a part 30 of the image field 32 acquired by the x-ray receiver 16a (FIG. 1), meaning the portion of the input screen surrounded by the blanking ring of the x-ray image intensifier, is shadowed. This shadowing is created by components of the lithotripter 1, significantly the coupler bellows 4, protrude into the beam path of the x-rays and lead to an intensified absorption of the x-ray radiation in the bellows wall and in the coupler medium. In order to prevent an overexposure of the area 34 lying outside of this shadowed part 30, to control the x-ray source 14a (FIG. 1) only the image data are used that originate from an image region 36, which lies in the normally exposed part 34. In the exemplary embodiment, the image region 36 is formed by two rectangular sub-regions 36a, 36b spatially separated from one another, one of which contains the center axis 20a, and thus the immediate surroundings of the focus F of the ultrasound shockwave, and the other is arranged outside of these immediate surroundings near the focus F, but still within the normally exposed part 34. The integral brightnesses of both sub-regions 36a and 36b are used to control the intensity of the x-ray radiation and thus the applied dose, but the data determined from the sub-region 36a are subject to a higher weighting than the data determined from the sub-region 36b. A modulation of the x-ray apparatus thus does not ensue based on the brightness of the total acquired image field 32 as in the prior art, but rather only based on the brightness of one or more predetermined image regions 36a or 36b that lie in the normally exposed part of the image field. This has the result that the shadowed part 30 already underexposed given a control of the x-ray system according to the prior art is additionally attenuated. In spite of this inventive underexposure, in order to be able to still diagnostically use the shadowed part 30 even without increasing the dose, for example to simplify the orientation, the data that are present in the shadowed part 30 are post-processed for contrast optimization in a digital image processing device independently of the normally exposed part 34.

FIG. 3 shows the imaging relationships in an “in-line” arrangement in which the x-ray receiver is arranged deviating from the positioning shown dashed in FIG. 1 in the region of the shockwave source. In this case, a larger part 30 of the total image field 32 is shadowed by the diaphragm 26. A likewise only weakly exposed part 40 of the image field 32 is not located between the image of the penetration opening 28 and the diaphragm 26. The normally exposed part 34 is located in the center of the image field 32 and limited by the penetration opening 28. To control the dose of the x-ray source 14b (FIG. 1), a central image region 42 is now used that contains the immediate surroundings around the focus F and the center axis 20b of the x-ray radiation as well as the center axis 24 of the shockwave. In this manner it is ensured that only the diagnostically relevant region within the focus region is correctly exposed while a calibrated underexposure is deliberately accepted in the remaining image zones.

Although modifications and changes may be suggested by those skilled in the art, it is the intention of the inventor to embody within the patent warranted hereon all changes and modifications as reasonably and properly come within the scope of his contribution to the art.

1. A method for controlling the radiation dose for generating an x-ray image of a focus region of a lithotripter, comprising the steps of:
generating an x-ray exposure of a focus region of a lithotripter by passing x-rays through the focus region and through at least one component of the lithotripter, said component producing a first region in said x-ray exposure shadowed by said component;
evaluating only a second region in said x-ray exposure, outside of said first region, to obtain an evaluation result; and
controlling a radiation dose of said x-rays, for producing subsequent x-ray exposures, using said evaluation result.

2. A method as claimed in claim 1 wherein the step of evaluating only said second region comprises evaluating an integral brightness of said second region to produce said evaluation result.

3. A method as claimed in claim 1 wherein said focus region has an immediate environment surrounding said focus region, and wherein said second region encompasses said environment.

4. A method as claimed in claim 1 comprising the additional step of lightening said first region by digital image processing.

5. A method as claimed in claim 1 comprising emitting said x-rays from an x-ray source having a center axis, and wherein said lithotripter has a center axis for shockwaves coinciding with said center axis of said x-ray source, and comprising gating said x-rays with a radiation diaphragm, and employing an image region as said second region that is disposed in a center of x-rays gated by said diaphragm.

6. A method as claimed in claim 5 comprising employing an image region as said second region having a circular shape.

7. A method as claimed in claim 1 wherein said lithotripter has a center axis along which said lithotripter generates shockwaves, and comprising generating said x-rays from an x-ray source having a center axis disposed at a non-zero angle relative to said center axis of said lithotripter.

8. A method as claimed in claim 7 comprising employing two spatially separated sub-regions in said x-ray exposure as said second region, both of said sub-regions being disposed outside of said first region.

9. A method as claimed in claim 8 comprising evaluating said second region by differently weighting said two sub-regions by differently weighting said two sub-regions.

10. A lithotripsy apparatus comprising:
a lithotripter having a focus region at which focused shockwaves are generated by said lithotripter, said lithotripter having at least one lithotripter component;
an x-ray source that emits x-rays for generating an x-ray exposure of said focus region, said x-rays also passing through said lithotripter component and said lithotripter component producing a first region in said x-ray exposure that is shadowed by said lithotripter component; and
an evaluation and control unit for evaluating only a second region of said x-ray exposure, disposed outside of said first region, for obtaining an evaluation result, and for controlling a dose of said x-rays for producing subsequent images of said focus region dependent on said evaluation result.

11. An apparatus as claimed in claim 10 comprising a switch for selectively deactivating control of said dose dependent on said evaluation result.

12. An apparatus as claimed in claim 10 wherein said evaluation and control unit evaluates an integral brightness of said second region for producing said evaluation result.

13. An apparatus as claimed in claim 10 wherein said focus region has an environment immediately surrounding said focus region, and wherein said evaluation and control unit employs an image region, as said second region, comprising said environment.

14. An apparatus as claimed in claim 10 wherein said evaluation and control unit lightens said first region by digital image processing.

15. An apparatus as claimed in claim 10 wherein said x-ray source has a center axis and wherein said lithotripter has a center axis coinciding with the center axis of said x-ray source, and comprising a diaphragm disposed in a path of said x-rays for gating said x-rays, and wherein said evaluation and control unit employs an image region in said x-ray exposure as said second region that is disposed in a center of the x-rays gated by said diaphragm.

16. An apparatus as claimed in claim 15 wherein said evaluation and control unit employs an image region as said second region having a circular shape.

17. An apparatus as claimed in claim 10 wherein said x-ray source has a center axis and wherein said lithotripter has a center axis disposed at a non-zero axis relative to said center axis of said x-ray source.

18. An apparatus as claimed in claim 17 wherein said evaluation and control unit employs two spatially separated sub-regions in said x-ray exposure as said second region, said two sub-regions being disposed outside of said first region.

19. An apparatus as claimed in claim 18 wherein said evaluation and control unit differently weights said two sub-regions for producing said evaluation result.
